(19) United States
(12) Patent Application Publication
(10) Pub. No.: US 2021/0205544 A1
(43) Pub. Date: Jul. 8, 2021

(54) NEEDLE GUARD

(71) Applicant: SHL MEDICAL AG, ZUG (CH)

(72) Inventor: Anders Holmqvist, Värmdö (SE)

(21) Appl. No.: 17/056,702

(22) PCT Filed: Aug. 23, 2019
(86) PCT No.: PCT/EP2019/072578

§ 371 Date: Nov. 18, 2020

(30) Foreign Application Priority Data
Sep. 5, 2018 (EP) 18192786.4

(51) Int. Cl. A61M 5/32

(52) U.S. Cl. CPC A61M 5/3202

(57) ABSTRACT

A needle guard for a medicament delivery device, where the needle guard has a body with a proximal end and a distal end, the proximal end having a generally circular opening of a predetermined diameter d. Proximally directed protrusions are arranged on a rim of the opening, and extend a predetermined length p from the rim of the opening such that a maximum intrusion depth i, of an object of a predetermined diameter D, into the opening, is reduced by p.

### CROSS REFERENCE TO RELATED APPLICATIONS

The present application is a U.S. National Phase Application pursuant to 35 U.S.C. § 371 of International Application No. PCT/EP2019/072578 filed Aug. 23, 2019, which claims priority to European Patent Application No. 18192786.4 filed Sep. 5, 2018. The entire disclosure contents of these applications are herewith incorporated by reference into the present application.

### TECHNICAL AREA

The present disclosure relates to a needle guard for a medicament delivery device, and more particularly to a safety device for a needle guard having a wide opening.

### BACKGROUND

Medicament delivery devices such as auto-injectors, safety syringes, and similar devices, often comprise a needle guard to prevent damage and contamination to the needle before use, and also to protect a user, or a person handling the device, from accidental needle sticks both before and after use. The needle guard is normally tubular, in the shape of a hollow sleeve. A proximal end has an opening, through which the needle will protrude when a needle penetration is performed.

Before use of the medicament delivery device, a cap often covers the needle guard and a proximal end of the device. A needle shield remover is commonly connected to the cap and is designed to protrude into the opening of the needle guard and engage with a needle shield in which the needle is hermetically embedded. Such a needle shield is normally either a Flexible Needle Shield (FNS) or a Rigid Needle Shield (RNS). The former comprises a soft rubber sheath, whereas the latter also comprises a rigid outer shell which encloses the rubber sheath within. In preparation for injection, a user removes the cap from the delivery device, resulting also in the removal of the needle shield due to the engagement between the needle shield remover and the needle shield.

Needle shields are usually standard components which come in a limited number of configurations and dimensions. It is normally a straightforward task to design a delivery device with regard to the needle shield. However, when the needle shield remover has to be re-designed it may have far-reaching consequences for the design of other components and mechanisms of the delivery device. It is especially disadvantageous if adaptation of the needle shield remover is required in a late stage of the development of the device, or even during production or after launch of a product.

One adaptation that might be necessary would be to increase a radius of the needle shield remover, for instance in order for the remover to be able to engage a wider needle shield. Consequently, the opening of the needle guard would also have to be made wider. But a wider opening means reduced protection of the needle inside, after the needle shield is removed, since it is easier to access the needle through a wide opening.

An obvious solution to the problem would be to increase the length of the needle guard so that an object intruding into the opening would have to travel further before contacting the needle. However, this requires the stroke length of the needle guard to be made longer because the needle has to travel further before skin penetration, while maintaining the same injection depth. With a longer stroke of the needle guard the activation mechanism needs to be re-designed to maintain the correct point of activation at a specified injection depth.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

Further, the term “longitudinal”, with or without “axis”, refers to a direction or an axis through the device or components thereof in the direction of the longest extension of the device or the component.

The term “lateral”, with or without “axis”, refers to a direction or an axis through the device or components thereof in the direction of the broadest extension of the device or the component. “Lateral” may also refer to a position to the side of a “longitudinally” elongated body.

In a similar manner, the terms “radial” or “transversal”, with or without “axis”, refers to a direction or an axis through the device or components thereof in a direction generally perpendicular to the longitudinal direction, e.g. “radially outward” would refer to a direction pointing away from the longitudinal axis.

In view of the foregoing, a general object of the present disclosure is to provide a needle guard for a medicament delivery device, which needle guard comprises a body having a proximal end and a distal end, the proximal end having a generally circular opening of a predetermined diameter d, proximally directed protrusions arranged on a rim of the opening, and wherein the proximally directed protrusions extend a predetermined length p from the rim of the opening such that a maximum intrusion depth i, of an object of a predetermined diameter D, into the opening, is reduced by p.

The protrusions thus enable a wider opening with maintained safety and without changing the stroke length of the needle guard because they will prevent an object from coming in contact with the needle, but will sink into the skin of a user to allow the needle penetration and injection to be performed at the correct injection depth.

In a further aspect of the disclosure the object is a generally spherical object and D is greater than d.

A spherical object of a certain diameter D is used to test the safety requirements of a needle guard. Therefore, the protrusions will fulfil the regulations if they reduce the intrusion depth of such a spherical object.

In yet an aspect of the disclosure the protrusions are arranged on the rim at the edge of the opening and configured such that a distance between proximal ends of protrusions on opposing sides of the opening generally equals d.

In practice, the position of the protrusions at the edge of the opening results in the needle guard, and its opening of a certain diameter d, being “extended” proximally without elongating the needle guard itself or its stroke length. This serves to keep the object away from the needle, i.e. to reduce the intrusion depth of the object.

In another aspect of the disclosure the protrusions are radially flexible and inclined radially inwards in a relaxed state such that a distance d′ between proximal ends of protrusions on opposing sides of the opening is less than d.

The protrusions may be slightly flexible so that they so that they collapse radially inwards when a cap and its needle shield remover is removed from the medicament delivery device. In this manner, the intrusion depth of the object may be reduced even further since the accessible opening is made smaller when the protrusions extend radially inwards.

### BRIEF DESCRIPTION OF DRAWINGS

In the following detailed description of the present disclosure, reference will be made to the accompanying drawings, of which

FIG. 1 is a perspective view of a prior art medicament delivery device

FIG. 2 is a perspective, cross-sectional view of a prior art medicament delivery device

FIG. 3 is a perspective, cross-sectional view of a medicament delivery device having a wider opening

FIG. 4 is a perspective, cross-sectional view of a medicament delivery device according to an embodiment of the present disclosure

FIG. 5 is cross-sectional view of a medicament delivery device having a wide opening

FIG. 6 is cross-sectional view of a medicament delivery device according to an embodiment of the present disclosure

FIG. 7 is cross-sectional view of a medicament delivery device according to an alternative embodiment of the present disclosure

### DETAILED DESCRIPTION

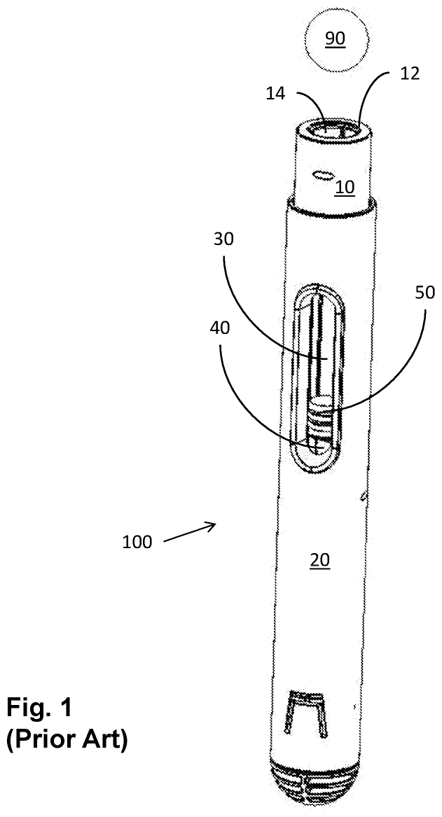

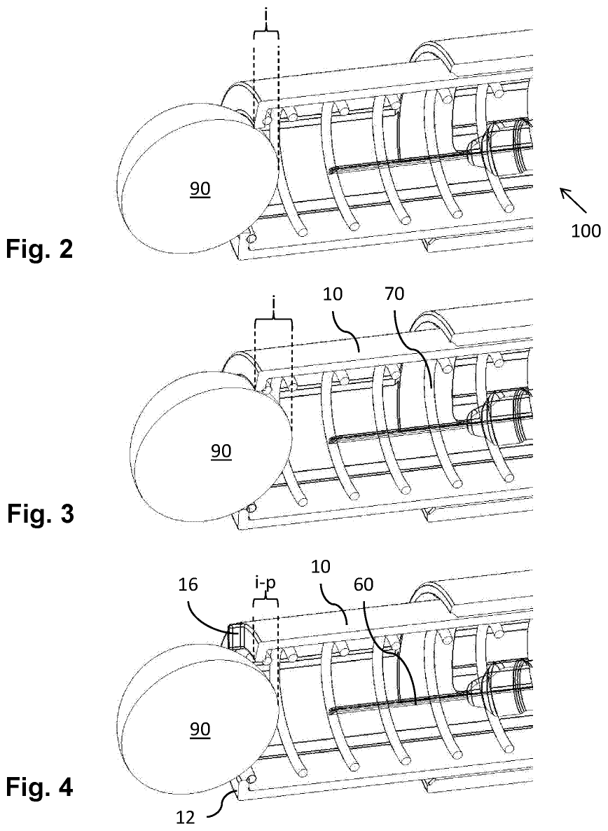

FIG. 1 shows a perspective view of a state of the art medicament delivery device 100. The medicament delivery device 100 has an axially elongated casing 20 which houses a medicament container 30 and a drive mechanism (not shown) arranged to drive a plunger rod 40 and a stopper 50 to pressurise a liquid medicament in the medicament container 30. The pressurised medicament is intended to be expelled through an injection needle 60 (FIGS. 2-4), which is applied to penetrate the skin of a user at a specific dose delivery site. The needle 60 is attached (or attachable) to a proximal end of the medicament container 30 and is protected by a needle guard 10, which is axially movable with respect to the casing 20 and with respect to the needle 60.

To perform the skin penetration and injection, the user holds the medicament delivery device 100 generally perpendicularly with respect to the skin, with the needle guard 10 touching the skin. The user pushes the medicament delivery device 100 proximally, causing the needle guard 10 to move distally in relation to the casing 20 and the needle 60. The relative movement exposes the needle to penetrate the skin and also activates the drive mechanism to pressurise and expel the medicament of the container 30. When the injection is complete, the user removes the medicament delivery device 100 from the dose delivery site, whereby a needle guard spring 70 (FIGS. 2-4) moves the needle guard 10 proximally, relative to the casing 20 and to the needle 60, into a position where the needle guard 10 is movably locked in order to protect the a person handling the used medicament delivery device 100 from accidental needle sticks.

Before use of the medicament delivery device 100, the needle guard 10, and consequently the needle 60, are protected by a removable cap (not shown) which covers the proximal end of the medicament delivery device 100. When the cap is in place it prevents the needle guard 10 from moving relative to the casing 20 and relative to the needle 60. Thus there is no risk of premature activation of the device or accidental needle sticks. The cap is removed by the user before injection.

Also shown in FIG. 1 is an object 90 of a predetermined object diameter D, which is used to test regulatory requirements of needle-protective features of a medicament delivery device 100. The object 90 is configured to mimic a fingertip and determines whether the needle guard 10 sufficiently shields the needle 60 of the medicament delivery device 100 in order to prevent accidental needle sticks.

The needle guard 10 may have a tubular shape and has a proximal end and a distal end, the proximal end having a generally circular opening 14 of a predetermined opening diameter d, through which the needle 60 may be exposed. The distal end extends into the casing 20 of the medicament delivery device 100, where it is configured to interact with the drive mechanism upon movement of the needle guard 10. The opening 14 is bordered by a rim 12, which rim 12 may be a generally proximally-facing annular ledge.

The object diameter D is greater than the opening diameter d such that the object is prevented from completely passing through the opening 14. However, a part of the object 90 may intrude into the opening 14, up to a certain intrusion depth i, measured from the proximal end of the needle guard 10. To comply with regulatory requirements, i.e. for the medicament delivery device 100 to be considered safe to use, the object 90 must not intrude more than a certain distance into the opening 14. In other words, the object 90 has to be stopped at a certain minimum distance from a sharp tip of the needle 60. For instance, when the object is placed on the opening 14, a distal end of the object must be at least 3.4 mm from the tip of the needle 60, i.e. a proximal end of the needle 60. Otherwise the medicament delivery device 100 will not pass the regulatory requirements.

FIG. 2 shows a cross-sectional view of a proximal end of the prior art medicament delivery device 100 of FIG. 1. The object 90 is positioned at the opening 14, in abutment with the needle guard 10, such that the intrusion depth i may be measured.

However, some therapeutic treatments require a different size of the medicament container 30, such as a greater diameter of the container, and or a larger needle shield as discussed in the Background section. The needle guard 10 would then also have to be adapted. One such adaptation may be the diameter d of the opening 14, which may have to be made larger. If the opening 14 is larger, accidental needle sticks will be more likely since the tip of the needle 60 is more easily accessible through the opening 14. The larger opening 14 will also result in that the regulatory test, using the object 90, will fail.

As shown in FIG. 3, the intrusion depth i is increased if the opening 14 is simply enlarged. The distance between the object 90 and the tip of the needle 60 will therefore be too short to pass the test.

An exemplary embodiment of the present disclosure is shown in FIG. 4. To be able to enlarge the opening 14, while maintaining the safety of the medicament delivery device 100, proximally directed protrusions 16 are arranged to extend a predetermined length p from the rim of the opening such that a maximum intrusion depth i, of an object of a predetermined object diameter D, into the opening, is reduced by p. FIG. 4 shows a reduced intrusion depth, as compared to the needle guard of FIG. 3, which lacks the protrusions 16.

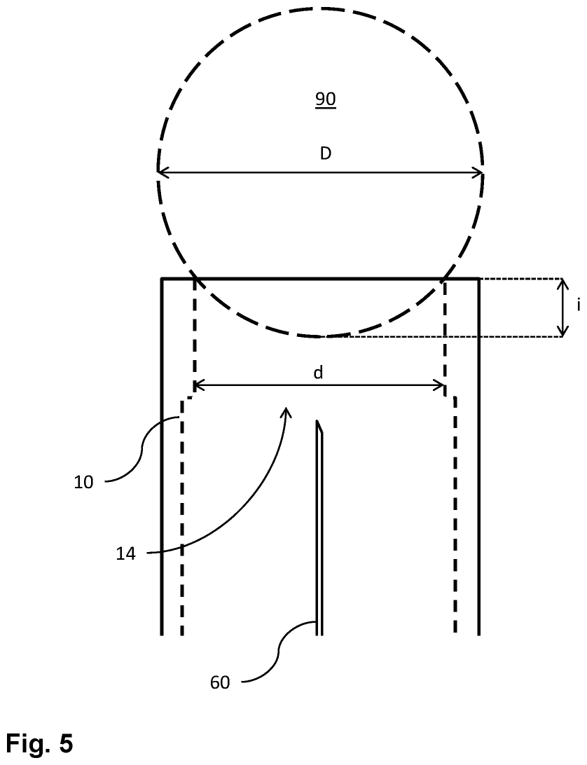

FIG. 5 is a conceptualised cross-sectional view of the device of FIG. 3. The opening 14 is enlarged to adapt to a larger medicament container. The intrusion depth i of the object 90 is consequently determined to be too large.

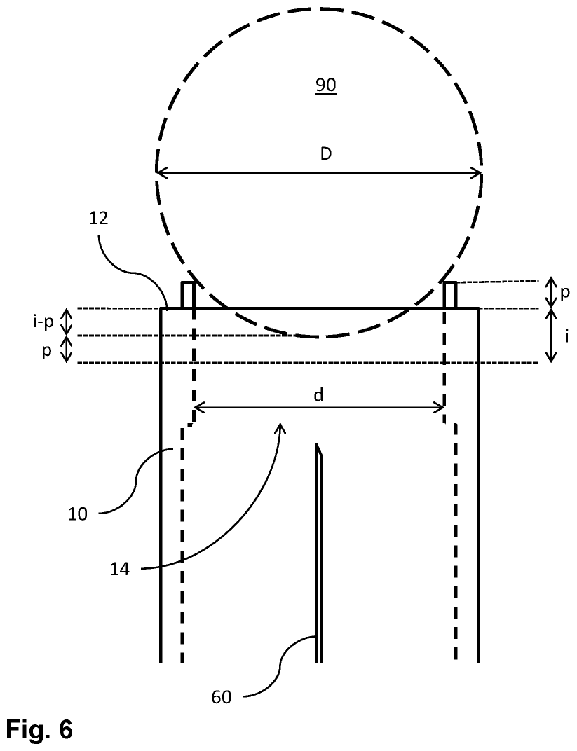

FIG. 6 is a conceptualised cross-sectional view of the device of FIG. 4, wherein the protrusions 16 have been arranged at the rim 12 of the opening 14. The protrusions extend proximally a distance p. Therefore, the intrusion depth i of the object 90 is reduced by p, allowing the needle guard to pass the test.

In order to offer the disclosed advantages, it may be preferable to arrange the protrusions 16 on the rim 12, i.e. at the edge of the opening 14, in order to provide a maximum depth reduction with regard to the extension p. The protrusions 16 should preferably further be configured such that a distance between proximal ends of protrusions on opposing sides of the opening generally equals the opening diameter d. In other words, it may be preferable to configure the protrusions 16 such that they do not extend radially inside the opening 14.

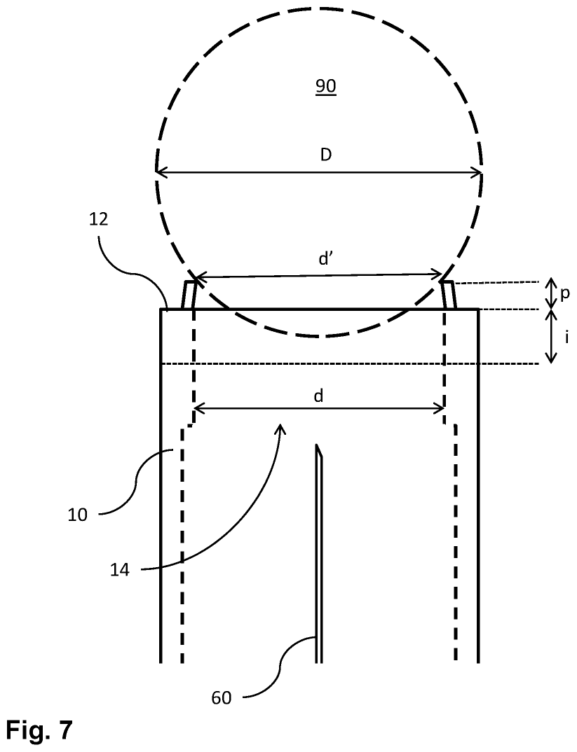

An alternative embodiment is shown in FIG. 7. The protrusions are configured to be radially flexible and inclined radially inwards in a relaxed state such that a distance d′ between proximal ends of protrusions on opposing sides of the opening is less than the opening diameter d. In this manner, the protrusions 16 will be kept in a tensioned state, axially parallel around the opening 14, when the cap (not shown) covers the proximal end of the medicament delivery device 100. However, when the cap is removed, the protrusions 16 are allowed to collapse radially inwards into the relaxed state. In this state the protrusions 16 extend radially inside the opening 14, such that the smaller diameter d′ between the proximal ends of the protrusions affects how far the object 90 may intrude into the opening 14. Thereby the intrusion depth is reduced further.

A suitable length p of the protrusions 16 may range between 0.5 mm-3 mm. More preferably the length p is 1 mm-2 mm. The length of the protrusions 16 should reduce the intrusion depth i of the object 90, but should not extend so far that the stroke length of the activation movement needs to be adjusted.

1-6. (canceled)

7. A needle guard for a medicament delivery device, which needle guard comprises,
a body having a proximal end and a distal end, the proximal end having a generally circular opening of a predetermined diameter d;
proximally directed protrusions arranged on a rim of the opening;
wherein the proximally directed protrusions extend a predetermined length p from the rim of the opening such that a maximum intrusion depth i, of an object of a predetermined diameter D, into the opening, is reduced by p.

8. A needle guard according to claim 7, wherein the object is a generally spherical object and D is greater than d.

9. A needle guard according to claim 7, wherein the protrusions are arranged on the rim at an edge of the opening, and configured such that a distance between proximal ends of protrusions on opposing sides of the opening generally equals d.

10. A needle guard according to claim 7, wherein the length p of the protrusions is in a range between about 0.5 mm and about 3 mm.

11. A needle guard according to claim 10, wherein the length p of the protrusions is approximately 1 mm to 2 mm

12. A needle guard according to claim 7, wherein the protrusions are radially flexible and inclined radially inwards in a relaxed state such that a distance d′ between proximal ends of protrusions on opposing sides of the opening is less than d.

13. A needle guard for a medicament delivery device, which needle guard comprises:
a body having a proximal end and a distal end, the proximal end having a rim with an edge portion and having a generally circular opening of a predetermined diameter d; and
a plurality of proximally directed protrusions arranged on the edge portion,
wherein each protrusion of the plurality of protrusions has a predetermined length p measured proximally from the rim of the opening such that a maximum intrusion depth i, of an object of a predetermined diameter D, into the opening is reduced by p.
wherein a distance between proximal ends of each protrusion on opposing sides of the opening generally equals the opening diameter d and do not extend radially inside the opening.
